SEQUENCE LISTING (2 sequences)

SEQ ID NO: 1 - DNA, 489 bp, Cercopithecus aethiops
  CDS (1)...(342)
atgagaatttcgaaaccacatttgagaagtatttccatccagtgctacctgtgtttacttctaaagagtcattttctaactgaagctggcattcatgtcttcattttgggctgtttcagtgcagggctccctaaaacagaagccaactgggtgaatgtaataagtgatttgaaaaaaattgaagatcttattcaatctatgcatattgatgctactttatatacagaaagtgatgttcaccccagttgcaaggtaacagcaatgaagtgctttctcttggagttgcaagttatttcacatgagtccggagatacagatattcatgatacagtagaaaatcttatcatcctagcaaacaacatcttgtcttctaatgggaatataacagaatctggatgcaaagaatgtgaggaactagaggaaaaaaatattaaagaatttttgcagagttttgtacatattgtccaaatgttcatcaacacttcttga

SEQ ID NO: 2 - DNA, 489 bp, Homo sapiens
  CDS (1)...(489)
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

(19) United States
(12) Patent Application Publication
(10) Pub. No.: US 2010/0086517 A1
(43) Pub. Date: Apr. 8, 2010

(54) Enhancement of B cell proliferation by IL-15

(75) Inventor: Yong Sung Choi, New Orleans, LA (US)

Correspondence Address: AMGEN INC., 1120 VETERANS BOULEVARD, SOUTH SAN FRANCISCO, CA, 94080, US

(73) Assignee: Ochsner Clinic Foundation, New Orleans, LA (US)

(21) Appl. No.: 12/456,436

(22) Filed: Jun. 15, 2009

Related U.S. Application Data
(63) Continuation of application No. 11/243,517, filed on Oct. 4, 2005, now abandoned.
(60) Provisional application No. 60/616,394, filed on Oct. 5, 2004.

(51) Int. Cl. A61K 38/20; A61K 39/395; A61K 38/21

(52) U.S. Cl. 424/85.2; 424/158.1; 424/133.1; 424/145.1; 424/85.7; 424/85.6; 424/85.5; 424/183.1

(57) ABSTRACT

Compositions and methods for modulating the growth, proliferation, and/or differentiation of B-cells in the germinal center are disclosed, and include use of IL-15 inhibitors, antagonists, and agonists. The compositions and methods find use in treating B-cell-related disorders, including neoplasms of the B-cell lineage.

### CROSS REFERENCE TO RELATED APPLICATION(S)

This application claims priority from U.S. Provisional Application No. 60/616,394 filed Oct. 5, 2004. The contents of all the above applications are incorporated herein by reference in their entirety.

### BACKGROUND OF THE INVENTION

1. Field of Invention

The present invention is in the field of IL-15-related modulation of B-cell growth and/or proliferation.

2. Description of the Related Art

Antigen-activated B cells proliferate and differentiate in the germinal center (“GC”). B-cells provide protection through the production of antibodies with optimal affinity against invading microorganisms (MacLennan, I. C. M. 1994. Annu. Rev. Immunology 12:117; Liu, Y.-J., et al. 1997. Immunology Rev. 156:111; Manser, T. 2004. J Immunology 172:3369). However, B-cells are also involved in numerous neoplastic conditions characterized by uncontrolled growth and multiplication of B-cell precursors. The GC provides a specialized microenvironment. Factors that control the vigorous proliferation of GC-B cells in this microenvironment are crucial for the expansion of a few initial clones as well as somatic hypermutation, a process through which a sufficient pool of diverse high affinity B cell receptors (“BCR's”) are obtained. Simultaneously, to ensure that the immune responses are not directed towards self-antigens, factors controlling the selection process within GC are also critical (Lindhout, E., et al. 1997. Immunology Today 18:573; Pulendran, B., et al. 1997. Immunology Today 18:27; Choe, J., et al. 1996. J. Immunology 157:1006). The signals received through a BCR known to be important for these GC reactions, have been investigated (Liu, Y.-J., et al. 1989. Nature 342:929; Kelsoe, G. 1996. Immunity 4:107; Haberman, A. M., et al. 2003. Nat Rev Immunology 3:757; Hande, S., et al. 1998. Immunity 8:189). The co-factors from the GC microenvironment, however, are not as clearly understood.

A major producer of GC microenvironmental factors is the follicular dendritic cell (FDC), which is present in lymphoid follicles and belongs to stromal cells of these organs (Haberman, A. M., et al. 2003. Nat Rev Immunology 3:757; Li, L., et al. 2002. Semin Immunology 14:259; van Nierop, K., et al. 2002. Semin Immunology 14:251; Lindhout, E., et al. 1995. Histochem J 27:167; Tew, J. G., et al. 1964. Immunology Rev 156:39). FDC's are initially known to retain antigens on their surface for a long time, and to present those native antigens to GC-B cells (Nossal, G. J. et al. 1964. Aust. J. Exp. Biol. 42:311; Kosco-Vilbois, M. H., et al. 1995. Current Topics of Microbiology in Immunology 201:69). FDCs are essential for GC-B cells to survive and proliferate in vitro upon stimulation with cytokines such as IL-2, IL-4 and IL-10 (Choe, J., et al. 1996. J. Immunology 157:1006; Zhang, X., et al. 2001. J. Immunology 167:49). Despite investigations on FDCs that have focused on their extraordinary capacities to support GC-B cell survival and proliferation via both direct cell-cell contact and secreted soluble factors (Tew, J. G., et al. 1990. Immunology Rev. 117:185; Grouard, G., et al. 1995. Journal of Immunology 155:3345; Kim, H.-S., et al. 1995. J. Immunology 155:1101; Kosco-Vilbois, M. H. 2003. Nat Rev Immunology 3:764), the factors identified to date have not been shown to replace the FDC effect completely (Lindhout, E., et al. 1995. Histochem J 27:167; Kim, H.-S., et al. 1995. J. Immunology 155:1101; van Eijk, M., et al. 1999. J Immunology 163:2478). Thus, there exists a need in the art for new compositions and methods to modulate GC-B cell survival and proliferation for treating B-cell related conditions including B cell-derived neoplasms, autoimmune disease, and B cell deficiencies.

### BRIEF DESCRIPTION OF THE DRAWINGS

FIG. 1. IL-15 is expressed in human tonsillar FDCs, but not in B cells. Cytospin preparations of human tonsillar FDC clusters were stained with goat polyclonal anti-IL-15 Ab (A and B: green), mouse anti-IL-15 mAb (D:green), corresponding control Abs (C and D-inset: green). Slides were co-stained with FDC-specific DRC-1 mAb for FDCs (A and C: red), anti-CD20 mAb for B cells (B: red) and DAPI for nucleus (D: blue). Original magnification ×400.

FIG. 2. FDC/HK cells express IL-15 on their surface bound to IL-15Rα. (A) Surface expression of IL-15 by FACS. Surface FACS staining with specific or control mAb was amplified with Flow-Amp kit (bold and dotted line, respectively). Competition experiments were performed to confirm specificity by incubating specific mAb with IL-15 (300 ng) for 30 min on ice prior to staining cells (thin line). (B) Change of surface IL-15 after acid stripping. FDC/HK cells were incubated in cold glycine buffer (pH 3.0) for 10 min on ice and then stained with specific Ab or isotype control Ab. (acid treatment: bold line; no treatment: thin line; isotype control: dotted line). (C) Expression of IL-15Rα mRNA in FDC/HK cells. RT-PCR for IL-15Rα and IL-2Rα (an internal control) was performed with same amount of FDC/HK cell mRNA under the same conditions.

FIG. 3. Membrane bound IL-15 on the FDC/HK surface is biologically active. Different numbers of FDC/HK cells (2 fold dilution from 2×10⁴ to none/well) were cultured in 96 well plates for 1 day and fixed with 1% paraformaldehyde. CTLL-2 cells (5×10³ cell/well) were cultured for 1 day on FDC/HK cell coated 96 well plates in triplicate in RPMI media containing 10% FCS, 1 U/ml of IL-2 and 2-ME. Cells were pulsed with 0.5 μCi of [³H] TdR (20 Ci/mM) for last 4 hours. [³H] TdR incorporation was measured by a liquid scintillation counter. Results are expressed as the mean cpm±SEM of triplicate cultures. (A) Proliferation of CTLL-2 cells in various number of FDC/HK cells added to the fixed number of CTLL-2 cells (None: 10% FCS RPMI medium control without coated FDC/HK; spn: FDC/HK culture supernatant). (B) Inhibition of enhanced CTLL-2 cell proliferation by specific anti-IL-15 mAb (10 μg/ml). Dotted line represents the cpm value of cultured CTLL-2 cells without FDC/HK cells or Ab. These results were reproduced in two independent experiments.

FIG. 4. GC B-cell expression of IL-15 and IL-2 receptors. (A) RT-PCR was performed with mRNAs from freshly isolated or cultured GC-B cells as described in Materials and Methods. ((+) control: plasmid containing respective genes; GCB d0: freshly isolated GCB cells; GC-B d4: GC-B cells were cultured for 4 days; DW: distilled water to serve as a negative control.) (B) FACS profiles of IL-15 binding assay. Freshly isolated GC-B cells and FDC/HK cells were incubated with a saturating dose of IL-15 (100 ng) for 30 min on ice, and then stained with anti-IL-15 mAb.

FIG. 5. IL-15 on FDC/HK cells increase GC-B cell recovery when cultured with FDC/HK cells and cytokines. (A) Viable cell recovery was decreased corresponding to the amount of added anti-IL-15 mAb. GC-B cells (2×10⁵ cell/well) were cultured in 24 well plates with FDC/HK cells (2×10⁴ cell/well, 5,000 Rad), CD40L (100 ng/ml), IL-2 (30 U/ml) and IL-4 (50 U/ml) with the indicated amount of specific mAb for 10 days. Cells were harvested at day 10 and counted by trypan blue exclusion. (B) The viable cell numbers were increased proportionally to the amount of added IL-15. Indicated amount of IL-15 was added to the GC-B cell cultures. IL-2 was not included in this experiment. Representative results from four separate experiments are presented.

FIG. 6. IL-15 enhances GC-B cell proliferation in vitro. Isolated GC-B cells were labeled with CFSE (5 μM/ml) and then were cultured for 6 days with IL-15(100 ng/ml), anti-IL-15 (10 μg/ml) or control mAb in the presence of FDC/HK cells and cytokine combinations. Harvested cells were counted and subjected to FACS analysis to measure the CFSE intensity. Results were analyzed with ModFit software. (A) Comparison of viable cell numbers. (B) Comparison of CFSE profiles of the recovered cells by percent in each division. (D: division)

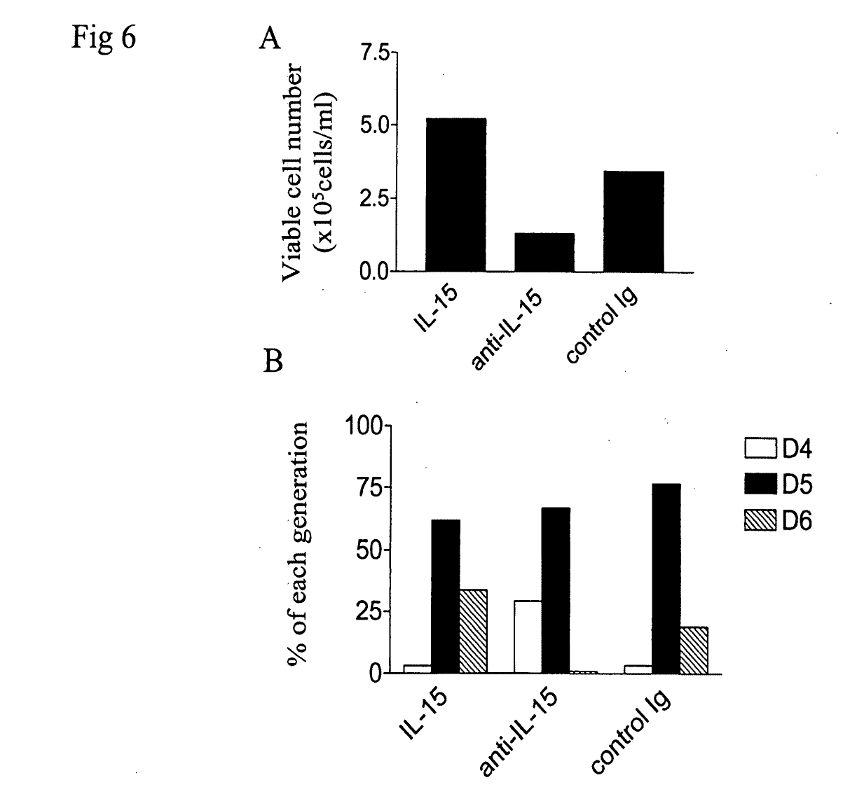

FIG. 7. IL-15 levels on the surface of FDC/HK are enhanced by GC-B cells or TNFα. FDC/HK cells were incubated for 3 days in 10% FCS IMDM media with various induction conditions as follows: Media alone (Media), IL-2, IL-4 and CD40L (24L); IL-2, IL-4 and CD40L with GC-B cells (24L+GC-B); TNF-α (10 ng/ml). Harvested cells were stained for FACS analysis. Numbers in the parenthesis represent MFI of each sample, which is calculated by subtracting control value from that of specific mAb (dotted line and solid line, respectively).

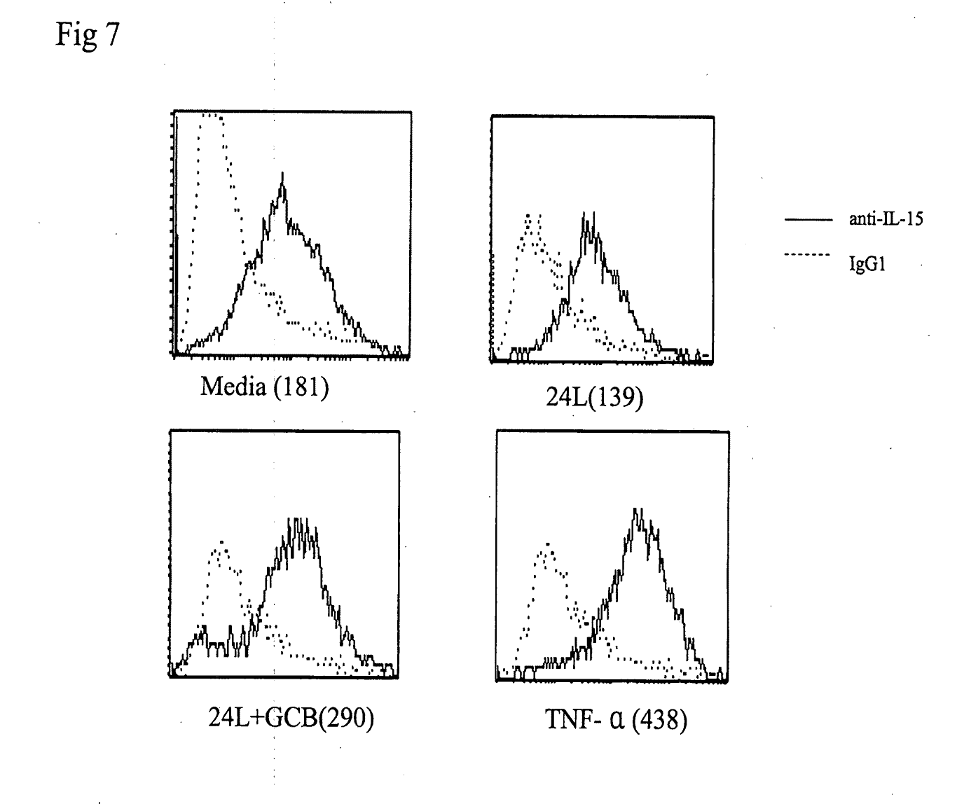

### SUMMARY OF THE INVENTION

The invention is directed to IL-15 antagonists and a method of using the antagonists for treatment of B-cell related human disease. In particular, such treatment includes inhibiting proliferation of neoplastic cells of B cell lineage. The IL-15 antagonists are effective by preventing IL-15 from transducing a signal to a cell through either the β- or γ-subunits of the IL-15 receptor complex, thereby antagonizing IL-15's biological activity towards B cells in the germinal centers.

The invention encompasses monoclonal antibodies that immunoreact with natural IL-15 and prevent signal transduction to the IL-15 receptor complex. The invention further encompasses humanized antibodies and human antibodies capable of inhibiting or preventing the binding of IL-15 to the β- or γ-subunit of the IL-15 receptor complex. The invention still further encompasses antagonists that block the IL-15Rα, including antibodies to this receptor subunit.

Antagonists according to the invention include soluble IL-15, and muteins of mature, or native, IL-15, wherein IL-15 has been mutagenized at one or more amino acid residues or regions that play a role in binding to the β- or γ-subunit of the IL-15 receptor complex. Such muteins prevent IL-15 from transducing a signal to the cells through either of the β- or γ-subunits of the IL-15 receptor complex, while maintaining the high affinity of IL-15 for the IL-15Rα. Typically, such muteins are created by additions, deletions or substitutions at key positions, for example, Asp⁵⁶ or Gln¹⁵⁶ of simian and human IL-15 as shown in SEQ ID NOS:1 and 2, respectively. It is believed that the Asp⁵⁶ affects binding with the β-subunit and that the Gln¹⁵⁶ affects binding with the γ-subunit of the IL-15 receptor complex.

Further included in the scope of the invention are modified IL-15 molecules that retain the ability to bind to the IL-15Rα, but have substantially diminished or no affinity for the β- and/or γ-subunits of the IL-15 receptor complex. Modified IL-15 molecules can take any form as long as the modifications are made in such a manner as to interfere with or prevent binding, usually by modification at or near the target binding site. Examples of such modified IL-15 molecules include natural IL-15 or a mutein of IL-1 5 that is covalently conjugated to one or more chemical groups that sterically interfere with the IL-15/IL-15 receptor binding. For example, natural IL-15 may contain site-specific glycosylation or may be covalently bound to groups such as polyethylene glycol (PEG), monomethoxyPEG (mPEG), dextran, polyvinylpyrrolidone (PVP), polyvinyl alcohol (PVA), poly amino acids such as poly-L-lysine or polyhistidine, albumin, gelatin at specific sites on the IL-15 molecule that can interfere with binding of IL-15 to the β- or γ-chains of the IL-15 receptor complex, while maintaining the high affinity of IL-15 for the IL-15Rα. By taking advantage of the steric hindrance properties of the group, binding to specific receptor subunits can be antagonized. Other advantages of conjugating chains of PEG to proteins such as IL-2, GM-CSF, asparagines, immunoglobulins, hemoglobin, and others are known in the art. For example, it is known that PEG prolongs circulation half-lives in vivo (see, Delgado, et al., Crit. Rev. Ther. Drug Carr. Syst., 9:249 (1992)), enhances solubility (see, Katre, et al., Proc. Natl. Acad. Sci., 84:1487 (1987)) and reduces immunogenicity (see, Katre, N. V., Immunology 144:209 (1990)).

The invention also is directed to the use of the antagonists in a method of treating a disease or condition in which a reduction in IL-15 activity on B cells is desired. Such diseases include leukemias and B cell lymphomas.

Accordingly, it is an object of the present invention to provide a method for treating B-cell malignancies using anti-IL-15 antibodies.

It is a further object of this invention to provide multimodal methods for treatment of B-cell malignancies in which doses of anti-IL-15 antibodies are supplemented with the administration of a therapeutic protein, such as an immunoconjugate or antibody fusion protein, or by a chemotherapeutic regimen.

These and other objects are achieved, in accordance with one embodiment of the present invention, by the provision of a method of treating a B-cell malignancy, comprising the step of administering to a subject having a B-cell malignancy an anti-IL-15 antibody and a pharmaceutically acceptable carrier.

### DETAILED DESCRIPTION OF THE INVENTION

### Introduction

According to the invention, IL-15 is produced by follicular dendritic cells (FDCs) and is presented on the surface of FDC/HK cells, being captured by IL-15 Rα and trans-presented to GC-B cells. The function of the IL-15 was studied on GC-B cells and FDCs using an in vitro experimental model that mimics the in vivo GC-reaction. GC-B cells do not express IL-15 Rα but do express the signal transduction complex IL-2/15 Rβ and Rγ. IL-15 presented on the membrane of FDC/HK cells is biologically active and co-stimulates proliferation of GC-B cells following CD40L stimulation. By identifying this mechanism, the invention provides new means for modulating normal and aberrant proliferation of GC-B cells.

### IL-15 Stimulation of GC B Cells

The discovery of the mechanism of GC-B cell stimulation through IL-15 indicates that B cell tumors of GC origin are particularly amenable to treatment using an inhibitor of IL-15-mediated B cell stimulation. Such inhibitors are discussed more fully herein. Examples of such tumors include precursor B-cell acute lymphoblastic leukemia (“ALL”) and lymphoma.

The data presented herein are important because it has been difficult to elucidate the role of B cells in some disease states. For example, previous study of the function of IL-15 in B cells has been hindered because in genetically modified mice, either eliminating IL-15 or forced expression model does not reveal evident differences in B cell responses compared to wild type mice (Kennedy, M. K., et al. 2000. J Exp Med 191:771; Lodolce, J. P., et al. 1998. Immunity 9:669; Marks-Konczalik, J., et al. 2000. Proc Natl Acad Sci USA 97:11445). IL-15 enhances proliferation and Ig secretion of human peripheral B cells (Armitage, R. J., et al. 1995. J Immunology 154:483. Bernasconi, N. L., et al. 2002. Science 298:2199. Litinskiy, M. B., et al. 2002. Nat Immunology 3:822.), inhibits apoptosis induced by anti-IgM (Bulfone-Paus, S., et al. 1997. Nat Med 3:1124.), and induces proliferation of malignant B cells (Tinhofer, I., et al. 2000. Blood 95:610. Trentin, L., et al. 1997. Leuk Lymphoma 27:35). However, the biologic function of IL-15 in GC reaction has not been demonstrated. In order to elucidate the role of IL-15 and to thereby develop compositions and methods for modulating this effect of IL-15, several studies are described herein. These studies reveal for the first time that follicular dendritic cells produce IL-15, and that IL-15 is presented on the surface of follicular dendritic cells. In this cell surface presentation form, the IL-15 enhances B lymphocyte proliferation by cellular contact. In contrast, the soluble form of IL-15 has no detectable effect on the target B lymphocytes. These discoveries were made through a series of experiments described below and in more detail in the Examples.

First, the cellular source of IL-15 within the GC was examined. Although IL-15 mRNA and small amounts of soluble IL-15 have been reported to be produced by in vitro-cultured FDC (Husson, H., et al. 2000. Cell Immunology 203:134.), the production of IL-15 by FDC at the protein level had not previously been demonstrated. IL-15 mRNA is almost ubiquitously expressed, and the production and secretion of protein is mainly controlled by complex and inefficient posttranslational mechanisms (Waldmann, T. A., et al. 1999. Annu Rev Immunology 17:19. Fehniger, T. A., et al. 2001. Blood 97:14.33, 34). Data disclosed herein reveal that FDCs produce IL-15 as shown by the immunofluorescent (“IF”) staining of freshly isolated FDC clusters. This in vivo observation was confirmed by data herein showing that a FDC cell line, FDC/HK cells, produced IL-15. IL-15 protein was detected on the surface of FDC/HK cells. The specificity of membrane bound IL-15 was confirmed by competition FACS analysis and by the blocking experiment of CTLL-2 bioassay. However, IL-15 was not detected by ELISA in the FDC/HK culture supernatant and this was further confirmed with the CTLL-2 assay.

Without being bound by a mechanism by which surface IL-15 expression is achieved, the complete loss of IL-15 staining after acid treatment, and enhanced binding after incubation with exogenous IL-15, strongly suggest a receptor-anchored mechanism rather than the presence of an alternative membrane form of IL-15. Although the possibility that failure to detect IL-15 after acid treatment resulted from denaturation of transmembrane form cannot be ruled out completely, expression of specific mRNA for IL-15Rα in FDC/HK cells also supports this mechanism.

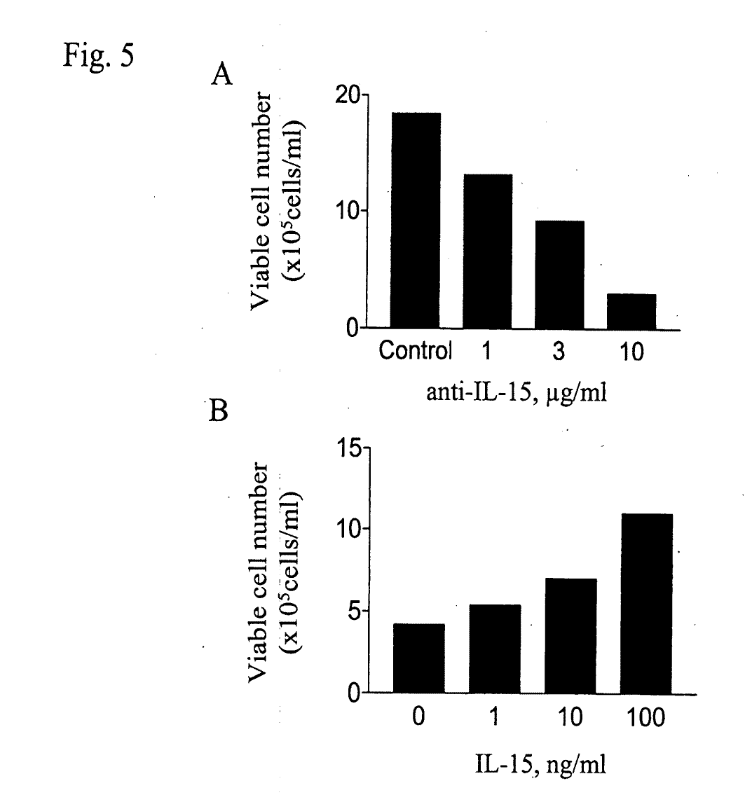

The biologic relevance of IL-15 signaling in the GC is demonstrated herein, by measuring the effect of IL-15 on GC-B cell proliferation by the removal or addition of IL-15. As shown in FIG. 5, GC-B cell growth decreased significantly in the presence of anti-IL-15 blocking mAb and was enhanced when IL-15 was added. Recovery of GC-B cells in the culture containing a saturating dose of IL-15 (100 ng/ml) was four fold higher than that of the culture where the activity of endogenous IL-15 was depleted by blocking mAb.

IL-15 is present on FDC in the GC in vivo and endogenous IL-15 from FDC/HK cells supported GC-B cell proliferation in vitro at levels comparable to, or more than, exogenous IL-2 alone when endogenous IL-15 was removed by blocking Ab (4.2×10⁵ in FIG. 5A left first bar vs. 2.9×10⁵ in FIG. 5B right end bar). Moreover, GC-B cells proliferated in the presence of IL-15, dividing faster than the cells cultured without IL-15. Together, these results indicate that IL-15 signaling may be one of the mechanisms responsible for the rapid proliferation of centroblasts in the GC in vivo.

Because IL-15 presentation by FDC may be an important trigger in the initiation of lymphomagenesis, immune modulation may be achieved by targeting the activity of IL-15 in GC-B cell proliferation. This mechanism also indicates that inhibiting IL-15 signaling in germinal centers provides a suitable treatment for B cell lymphomas.

Conditions amenable to treatment by modulating IL-15 stimulation of B cells. B cells stimulated in the germinal centers can take a variety of developmental routes, some of which are normal, and some of which are pathological. The route selected for modulation by the methods of the invention, and the related medical condition, will determine whether an antagonist of IL-15, or a stimulator or agonist, should be employed. Conditions and disorders suitable for modulation according to methods described herein are listed below, and subsequently discussed in more detail: B cell lymphomas; leukemias of B cell origin; antibody immunodeficiency disorders; combined antibody-mediated (B cell) and cell-mediated (T cells) immunodeficiency disorders; and autoimmune disease.

In addition to these disorders, the invention also provides for treatment of any other disorder in which modulation of B cell stimulation via IL-15 in the germinal center plays a role.

B cell lymphomas. Lymphomas that are suitable for treatment by inhibiting IL-15-mediated proliferation of GC-B-cells include non-Hodgkin's lymphoma, which is derived from germinal center B-cells with non-productive immunoglobulin gene rearrangements; B-cell lymphomas (the most common non-Hodgkin's lymphomas in the United States); Hodgkin's lymphoma; small lymphocytic lymphoma (SLL/CLL); mantle cell lymphoma (MCL); follicular lymphoma; marginal cell lymphoma, which includes extranodal, or MALT, lymphoma; nodal, or monocytoid B-cell, lymphoma; splenic lymphoma; diffuse large cell lymphoma; Burkitt's lymphoma; high grade Burkitt-like lymphoma; and lymphoblastic lymphoma. Also included is diffuse large cell lymphoma, which may exist as one of at least six morphological variants (centroblastic, immunoblastic, T-cell histocyte-rich, lymphomatoid granulomatosis type, anaplastic, and plasma blastic), and one of at least three subtypes (mediastinal, or thymic; primary effusion lymphoma; and intravascular (previously referred to as malignant angioendotheliomatosis).

Hodgkin's lymphoma (Hodgkin disease) itself is classified into several categories under the WHO classification system: nodular lymphocyte-predominant Hodgkin lymphomas; and classic Hodgkin lymphomas, including nodular sclerosis Hodgkin lymphoma; lymphocyte-rich Hodgkin lymphoma; mixed cellularity Hodgkin lymphoma; and lymphocyte depletion Hodgkin lymphoma.

B Cell Proliferative Disorders. B cell proliferative disorders suitable for treatment described herein include post-transplant lymphoproliferative disorders (PTLD's). Early lesions of this disorder include plasmacytic hyperplasia, atypical lymphoid hyperplasia, and infectious mononucleosis-like PTLD. Other categories include polymorphic PTLD and monomorphic PTLD. Although these conditions often regress spontaneously or with reduction of post-transplant immunosuppression, they can be fatal.

Antibody (B cell) Immunodeficiency Disorders. Antibody disorders associated with deficient B cell differentiation and proliferation are amenable to treatment by enhancing IL-15-induced GC-B cell proliferation. These disorders include: X-linked hypogammaglobulinemia (congenital hypogammaglobulinemia); transient hypogammaglobulinemia of infancy; common, variable, unclassifiable immunodeficiency (acquired hypogammaglobulinemia); immunodeficiency with hyper-IgM; neutropenia with hypogammaglobulinemia; polysaccharide antigen unresponsiveness; selective IgA deficiency; selective IgM deficiency; selective deficiency of IgG subclasses; secondary B cell immunodeficiency associated with drug, protein-losing conditions; and X-linked lymphoproliferative disease.

Combined antibody-mediated (B cell) and cell-mediated (T cell) immunodeficiency disorders. Enhancement of the B cell component of these diseases can be accomplished as discussed above for B cell immunodeficiency disorders. Such diseases include: Severe combined immunodeficiency disease (autosomal recessive, X-linked, sporadic); cellular immunodeficiency with abnormal immunoglobulin synthesis (Nezelof's syndrome); immunodeficiency with ataxia-telangiectasia; immunodeficiency with eczema and thrombocytopenia (Wiskott-Aldrich syndrome); immunodeficiency with thymoma; immunodeficiency with short-limbed dwarfism; immunodeficiency with adenosine deaminase deficiency; immunodeficiency with nucleoside phosphorylase deficiency; biotin-dependent multiple carboxylase deficiency; graft-versus-host (GVH) disease; and acquired immunodeficiency syndrome (AIDS).

Autoimmune Disorders. B cells produce immunoglobulins, and play a critical role in antibody mediated autoimmunity. B cell deficient mice, produced by administration of anti-μ antibodies beginning at birth, were resistant to some autoimmune diseases, including experimental autoimmune encephalitis, and spontaneous insulin dependent diabetes. (Looney, Ann. Rheum. Dis. 61:863). Mice genetically deficient in B cells may also have a lower tendency to develop autoimmune disease. For example, in B cell deficient mice, auto-antibodies were absent, and the increase in T cells in lymphoid organs was prevented, as described by Chan et al., J. Immunol. 160:51-59 (1998). Depletion of B cells using anti-CD-20 antibodies may be of therapeutic benefit in treating autoimmune diseases such as autoimmune cytopenias. In addition to decreasing the potentially pathogenic antibodies, the reduction in B cells can modulate the T cell activity, further decreasing the immune response to auto-antigens. (Gorozny et al., Arthritis Res. Ther. 5:131-135, 2003.)

There is substantial evidence of a critical role for B cells in the induction and progress of autoimmune disease. Thus, the methods of the invention find use in treating autoimmune disease by inhibiting B cell development, and hence decreasing or preventing altogether the levels of pathological auto-antigens in the patient. Autoimmune diseases amenable to such treatment include nervous system diseases such as multiple sclerosis, myasthenia gravis, autoimmune neuropathies including Guillain-Barre, and autoimmune uveitis. Gastrointestinal system diseases include Crohn's Disease, ulcerative colitis, primary biliary cirrhosis, and autoimmune hepatitis. Diseases affecting the blood include autoimmune hemolytic anemia, pernicious anemia, and autoimmune thrombocytopenia; diseases affecting the blood vessels include temporal arteritis, anti-phospholipid syndrome, vasculitis including Wegener's granulomatosis, and Bechet's Diseases. Diseases of the endocrine glands include Type I or immune-mediated diabetes mellitus, Grave's Disease, Hashimoto's thyroiditis, autoimmune oophoritis and orchitis, and autoimmune disease of the adrenal gland. Skin diseases include psoriasis, dermatitis herpetiformis, pemphigus vulgaris, and vitiligo. Finally, diseases affecting multiple organs, also called diseases of the connective tissue, include rheumatoid arthritis, systemic lupus erythematosus, scleroderma, polymyositis and dermatomyositis, spondyloarthropathies including ankylosing spondyltisi, and Sjogren's syndrome.

B cell leukemias. Acute lymphocytic leukemia (ALL) is also amenable to treatment with inhibitors of IL-15 stimulation of B cells. ALL is a malignant cell disorder caused by the clonal proliferation of lymphoid precursor cells with arrested maturation. ALL can originate in cells of B or T lineage, causing B cell leukemia, T cell leukemia, and leukemias of mixed cell lineage. Both B cell leukemia, and leukemia of mixed cell lineage, are appropriate for treatment using the methods herein. In adults, ALL constitutes about 20% of leukemias (Brincker, H., Scand. J. Maematol. 29:241-249, 1982), and about 1-2% of all cancers (Boring, C. C. et al., Cancer J. Clin. 44:7-16, 1994). B cell related ALL classifications include early pre-B-cell ALL; pre-B-cell ALL; transitional pre-B-cell ALL; and mature B-cell ALL. Mature B-cell ALL represents a leukemic phase of Burkitt's lymphoma (Magrath, I. T. et al., Leukemia Res. 4:33-59, 1979).

IL-15 antagonists. IL-15 antagonists of the invention that can modulate IL-15 effects in the germinal center include (a) soluble IL-15, wherein the soluble IL-15 is expected to block the binding of IL-15-Rα-attached IL-15 to the IL-15 β- and/or γ-receptor subunits of germinal center B cells; (b) a mutein of mature, or native, IL-15 capable of binding to the α-subunit of the IL-15 receptor and incapable of transducing a signal through the β- and/or γ-subunits of the IL-15 receptor complex; (c) a monoclonal antibody against IL-15 that prevents IL-15 from effecting signal transduction through the β-and/or γ-subunits of the IL-15 receptor complex; and (d) an IL-15 molecule that is covalently bonded with a chemical group that interferes with the ability of IL-15 to effect a signal transduction through either the β- or γ-subunits of the IL-15 receptor complex, but does not interfere with IL-15 binding to IL-15Rα. Also included in the scope of the present invention are polynucleotides that encode the muteins described above.

“IL-15 mutein” or “muteins of IL-15” refer to the mature, or native, simian IL-15 molecules having the sequence of amino acids 49-162 of SEQ ID NO:1 or human IL-15 molecules having the sequence of amino acids 49-162 of SEQ ID NO:2, that have been mutated in accordance with the invention in order to produce an antagonist of IL-15. Such IL-15 muteins are capable of binding to the IL-15Rα subunit, and are incapable of transducing a signal through the β- or γ-subunits of the IL-15 receptor complex.

Human or simian L-15 can be obtained according to the procedures described by Grabstein et al., Science, 264:965 (1994), which has been incorporated herein by reference, or by conventional procedures such as polymerase chain reaction (PCR). There are many possible mutations of IL-15 that can produce antagonists. Such mutations can be made at specific amino acid sites believed to be responsible for β- or γ-subunit signaling; or mutations can be made over entire regions of IL-15 that are considered necessary for β- or γ-subunit signaling. Typically, mutations may be made as additions, substitutions or deletions of amino acid residues. Preferably, substitution and deletion muteins are preferred with substitution muteins being most preferred.

It is believed that the Asp⁵⁶ affects binding with the β-subunit and that the Gln¹⁵⁶ affects binding with the γ-subunit of the IL-15 receptor complex. Adding or substituting other naturally-occurring amino acid residues near or at sites Asp⁵⁶ and Gln¹⁵⁶ can affect the binding of IL-15 to either or both of the β- or γ-subunits of the IL-15 receptor complex. For example, removing the negatively-charged aspartic acid residue and replacing it with another negatively-charged residue may not be as effective at blocking receptor binding as if the aspartic acid were replaced with a positively-charged amino acid such as arginine, or uncharged residues such as serine or cysteine.

Recombinant production of an IL-15 mutein first requires isolation of a DNA clone (i.e., cDNA) that encodes an IL-15 mutein. cDNA clones are derived from primary cells or cell lines that express mammalian IL-15 polypeptides. First total cell mRNA is isolated, then a cDNA library is made from the mRNA by reverse transcription. A cDNA clone may be isolated and identified using the DNA sequence information provided herein to design a cross-species hybridization probe or PCR primer as described above. Such cDNA clones have the sequence of SEQ ID NO:1 and SEQ ID NO:2. Recombinant production of IL-15 muteins is described in U.S. Pat. No. 6,177,079, incorporated hereby reference.

Equivalent DNA constructs that encode various additions or substitutions of amino acid residues or sequences, or deletions of terminal or internal residues or sequences not needed for activity, are useful for the methods of the invention. For example, N-glycosylation sites in IL-15 can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. The simian IL-15 protein comprises two such triplets, at amino acids 127-129 and 160-162 of SEQ ID NO:1. The human IL-15 protein comprises three such triplets, at amino acids 119-121, 127-129 and 160-162 of SEQ ID NO:2. Appropriate substitutions, additions or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Known procedures for inactivating N-glycosylation sites in proteins include those described in U.S. Pat. No. 5,071,972 and EP 276,846, hereby incorporated by reference.

Recombinant expression vectors include synthetic or cDNA-derived DNA fragments encoding an IL-15 mutein. The DNA encoding an IL-15 mutein is operably linked to a suitable transcriptional or translational regulatory or structural nucleotide sequence, such as one derived from mammalian, microbial, viral or insect genes. Examples of regulatory sequences include, for example, a genetic sequence having a regulatory role in gene expression (e.g., transcriptional promoters or enhancers), an optional operator sequence to control transcription, a sequence encoding suitable mRNA ribosomal binding sites, and appropriate sequences that control transcription and translation initiation and termination. Nucleotide sequences are operably linked when the regulatory sequence functionally relates to the structural gene. For example, a DNA sequence for a signal peptide (secretory leader) may be operably linked to a structural gene DNA sequence for an IL-15 mutein if the signal peptide is expressed as part of a precursor amino acid sequence and participates in the secretion of an IL-15 mutein. Further, a promoter nucleotide sequence is operably linked to a coding sequence (e.g., structural gene DNA) if the promoter nucleotide sequence controls the transcription of the structural gene nucleotide sequence. Still further, a ribosome binding site may be operably linked to a structural gene nucleotide coding sequence (e.g. IL-15 mutein) if the ribosome binding site is positioned within the vector to encourage translation.

Suitable host cells for expression of an IL-15 mutein include prokaryotes, yeast or higher eukaryotic cells under the control of appropriate promoters. Prokaryotes include gram negative or gram positive organisms, for example E. coli or bacilli. Suitable prokaryotic host cells for transformation include, for example, E. coli, Bacillus subtilis, Salmonella typhimurium, and various other species within the genera Pseudomonas, Streptomyces, and Staphylococcus. Examples of suitable host cells also include yeast such as S. cerevisiae, a mammalian cell line such as Chinese Hamster Ovary (CHO) cells, or insect cells. Cell-free translation systems could also be employed to produce an IL-15 mutein using RNAs derived from the DNA constructs disclosed herein. Appropriate cloning and expression vectors for use with bacterial, insect, yeast, and mammalian cellular hosts are described, for example, in Pouwels et al. Cloning Vectors: A Laboratory Manual, Elsevier, N.Y., 1985.

When an IL-15 mutein is expressed in a yeast host cell, the nucleotide sequence (e.g., structural gene) that encodes an IL-15 mutein may include a leader sequence. The leader sequence may enable improved extracellular secretion of translated polypeptide by a yeast host cell.

Methods of preparing and purifying IL-15 muteins are described in U.S. Pat. No. 6,177,079, incorporated herein by reference. Preferably, a mutein of IL-15 is used wherein at least one of the amino acid residues Asp⁵⁶ or Gln¹⁵⁶ of IL-15 (simian IL-15 having the sequence of amino acid residues 49-162 shown in SEQ ID NO:1 or human IL-15 having the sequence of amino acid residues 49-162 shown in SEQ ID NO:2) is deleted or substituted with a different naturally-occurring amino acid residue. Any combination of substitutions and/or deletions can be made. For example, Asp⁵⁶ can be deleted while Asp⁵⁶ is substituted with any other amino acid, or both Asp⁵⁶ and Gln¹⁵⁶ are each substituted with the same or different amino acid moiety. Further, Asp⁵⁶ can be substituted with any amino acid while Gln¹⁵⁶ is deleted. Generally, substitution muteins are preferred, and more preferred are those that do not severely affect the natural folding of the IL-15 molecule. Substitution muteins preferably include those wherein Asp⁵⁶ is substituted by serine or cysteine; or wherein Gln¹⁵⁶ is substituted by serine or cysteine, or wherein both Asp⁵⁶ and Gln¹⁵⁶ are each substituted with a serine or cysteine. Examples of deletion muteins include those wherein Asp⁵⁶ and Gln¹⁵⁶ of mature IL-15 are both deleted; wherein only Asp⁵⁶ is deleted; or wherein only Gln¹⁵⁶ is deleted. It is possible that other amino acid residues in the region of either Asp⁵⁶ and Gln¹⁵⁶ can be substituted or deleted and still have an effect of preventing signal transduction through either or both of the β or γ subunits of the IL-15 receptor complex. Therefore, the invention further utilizes muteins wherein amino acid residues within the region of Asp⁵⁶ and Gln¹⁵⁶ are either substituted or deleted, and that possess IL-15 antagonist activity. Such muteins can be made using the methods described herein and can be assayed for IL-15 antagonist activity using conventional methods. Further description of a method that can be used to create the IL-15 muteins utilized in the invention is provided in U.S. Pat. No. 6,177,079.

The mature IL-15 polypeptides utilized herein (mature simian IL-15 comprising the sequence of amino acids 49-162 of SEQ ID NO:1 and mature human IL-15 having the sequence of amino acid residues 49-162 shown in SEQ ID NO:2), as well as the IL-15 muteins, may be modified by forming covalent or aggregative conjugates with other chemical moieties. Such moieties can include PEG, mPEG, dextran, PVP, PVA, polyamino acids such as poly-L-lysine or polyhistidine, albumin and gelatin at specific sites on the IL-15 molecule that can interfere with binding of IL-15 to the β- or γ-chains of the IL-15 receptor complex, while maintaining the high affinity of IL-15 for the IL-I5Rα. Additionally, IL-15 can be specifically glycosylated at sites that can interfere with binding of IL-15 to the β- or γ-chains of the IL-15 receptor complex, while maintaining the high affinity of IL-15 for the IL-15Rα. Preferred groups for conjugation are PEG, dextran and PVP. Most preferred for use in the invention is PEG, wherein the molecular weight of the PEG is preferably between about 1,000 to about 20,000. A molecular weight of about 5000 is preferred for use in conjugating IL-15, although PEG molecules of other weights would be suitable as well. A variety of forms of PEG are suitable for use in the invention. For example, PEG can be used in the form of succinimidyl succinate PEG (SS-PEG) which provides an ester linkage that is susceptible to hydrolytic cleavage in vivo, succinimidyl carbonate PEG (SC-PEG) which provides a urethane linkage and is stable against hydrolytic cleavage in vivo, succinimidyl propionate PEG (SPA-PEG) provides an ether bond that is stable in vivo, vinyl sulfone PEG (VS-PEG) and maleimide PEG (Mal-PEG) all of which are commercially available from Shearwater Polymers, Inc. (Huntsville, Ala.). In general, SS-PEG, SC-PEG and SPA-PEG react specifically with lysine residues in the polypeptide, whereas VS-PEG and Mal-PEG each react with free cysteine residues. However, Mal-PEG is prone to react with lysine residues at alkaline pH. Preferably, SC-PEG and VS-PEG are preferred, and SC-PEG is most preferred due to its in vivo stability and specificity for lysine residues.

The PEG moieties can be bonded to IL-15 in strategic sites to take advantage of PEG's large molecular size. As described in U.S. Pat. No. 6,177,079, PEG moieties can be bonded to IL-15 by utilizing lysine or cysteine residues naturally occurring in the protein or by site-specific PEGylation. One method of site specific PEGylation is through methods of protein engineering wherein cysteine or lysine residues are introduced into IL-15 at specific amino acid locations. The large molecular size of the PEG chain(s) conjugated to IL-15 is believed to block the region of IL-15 that binds to the β- and/or γ-subunits but not the α-subunit of the IL-15 receptor complex. Conjugations can be made by addition reaction wherein PEG is added to a basic solution containing IL-15. Typically, PEGylation is carried out at either (1) about pH 9.0 and at molar ratios of SC-PEG to lysine residue of approximately 1:1 to 100:1, or greater, or (2) at about pH 7.0 and at molar ratios of VS-PEG to cysteine residue of approximately 1:1 to 100:1, or greater.

Alternatively, an antagonist according to the invention can take the form of a monoclonal antibody against IL-15 that interferes with the binding of IL-15 to any of the β- or γ-subunits of the IL-15 receptor complex. Within one aspect of the invention, IL-15, including derivatives thereof, as well as portions or fragments of these proteins such as IL-15 peptides, can be used to prepare antibodies that specifically bind to IL-15. Within the context of the invention, the term “antibodies” should be understood to include polyclonal antibodies, monoclonal antibodies, fragments thereof such as F(ab′)2 and Fab fragments, as well as recombinantly produced binding partners. The affinity of a monoclonal antibody or binding partner may be readily determined by one of ordinary skill in the art (see Scatchard, Ann. N.Y. Acad. Sci., 51: 660-672 (1949)). Specific examples of such monoclonal antibodies include antibodies produced by the clones designated as M110, M111 and M112, which are IgG1 monoclonal antibodies. Hybridomas producing monoclonal antibodies M110, M111 and M112 were deposited with the American Type Culture Collection, Rockville, Md., USA (ATCC) on Mar. 13, 1996 and assigned accession numbers HB-12061, HB-12062, and HB-12063, respectively. All deposits were made according to the terms of the Budapest Treaty.

In general, monoclonal antibodies against IL-15 can be generated as described in U.S. Pat. No. 6,177,079, using the following procedure. Briefly, purified IL-15, a fragment thereof, synthetic peptides or cells that express IL-15 can be used to generate monoclonal antibodies against IL-15 using techniques known per se, for example, those techniques described in U.S. Pat. No. 4,411,993. Mice are immunized with IL-15 as an immunogen emulsified in complete Freund's adjuvant or RIBI adjuvant (RIBI Corp., Hamilton, Mont.), and injected in amounts ranging from 10-100 μg subcutaneously or intraperitoneally. Ten to twelve days later, the immunized animals are boosted with additional IL-15 emulsified in incomplete Freund's adjuvant. Mice are periodically boosted thereafter on a weekly to bi-weekly immunization schedule. Serum samples are periodically taken by retro-orbital bleeding or tail-tip excision to test for IL-15 antibodies by dot blot assay, ELISA (Enzyme-Linked Immunosorbent Assay) or inhibition of IL-15 activity on CTLL-2 cells.

Following detection of an appropriate antibody titer, positive animals are provided an additional intravenous injection of IL-15 in saline. Three to four days later, the animals are sacrificed, spleen cells harvested, and spleen cells are fused to a murine myeloma cell line, for example, NS1 or P3x63Ag8.653 (ATCC CRL 1580). Fusions generate hybridoma cells, which are plated in multiple microtiter plates in a HAT (hypoxanthine, aminopterin and thymidine) selective medium to inhibit proliferation of non-fused myeloma cells and myeloma hybrids.

The hybridoma cells are screened by ELISA for reactivity against purified IL-15 by adaptations of the techniques disclosed in Engvall et al., Immunochem. 8:871, 1971 and in U.S. Pat. No. 4,703,004. A preferred screening technique is the antibody capture technique described in Beckmann et al., (J. Immunology 144:4212, 1990). Positive hybridoma cells can be injected intraperitoneally into syngeneic Balb/c mice to produce ascites containing high concentrations of anti-IL-15 monoclonal antibodies. Alternatively, hybridoma cells can be grown in vitro in flasks or roller bottles by various techniques. Monoclonal antibodies produced in mouse ascites can be purified by ammonium sulfate precipitation, followed by gel exclusion chromatography. Alternatively, affinity chromatography based upon binding of antibody to protein A or protein G can also be used, as can affinity chromatography based on binding to IL-15.

Other antibodies can be prepared utilizing the disclosure and material incorporated by reference provided herein, and are useful for the present invention. Procedures used to generate humanized antibodies can be found in U.S. Pat. Nos. 4,816,567, 6,500,931 and WO 94/10332, all of which are incorporated by reference herein. Procedures to generate human antibodies, such as use of mice or other mammals expressing polynucleotides encoding human antibody proteins, are disclosed in, for example, U.S. Pat. Nos. 6,075,181; 6,111,166; 6,673,986; 6,680,209; and 6,682,726, all of which are incorporated by reference herein. Procedures to generate microbodies can be found in WO 94/09817; and additional procedures to generate transgenic antibodies can be found in GB 2 272 440, all of which are incorporated herein by reference.

Additional antagonists of use in the methods of the invention include antagonists to the IL-15Rα subunit. Such antagonists are disclosed in, for example, U.S. Pat. No. 5,591,630, which is incorporated by reference herein.

To determine which monoclonal antibodies are antagonists, use of a screening assay is preferred. A CTLL-2 proliferation assay is preferred for this purpose. See, Gillis and Smith, Nature 268:154 (1977), which is incorporated herein by reference. Briefly, antagonist activity of monoclonal antibodies, PEGylated IL-15 and IL-15 muteins can be assessed using a modified CTLL-2 cell ³H-Thymidine incorporation assay (Gillis, et al., Id.). Serial dilutions of antagonist can be made in 96-well flat-bottom tissue culture plates (Costar, Cambridge, Mass.) in DMEM medium (supplemented with 5% FCS, NEAA, NaPyruvate, HEPES pH 7.4, 2-me, PSG) at a final volume of 50 μl. A sub-optimal amount of IL-15 (final concentration of 20-40 pg/ml) then is added to all assay wells (5 μl/well) after serial dilution of samples and prior to addition of cells. Washed CTLL-2 cells are added (about 2000 per well in 50 μl) and the plates are incubated for 24 hours at 37° C. in a humidified atmosphere of 10% CO² in air. This was followed by a five hour incubation with 0.5 μCi of ³H-Thymidine (25 Ci/mMol, Amersham, Arlington Heights, Ill.). The cultures then are harvested on glass fiber filters and counted by avalanche gas ionization either on a multidetector direct beta counter (Matrix 96, Packard Instrument Company, Meridien, Conn.) or on a beta scintillation counter. The counts per minute (CPM) generated by the assay are converted to percent inhibition and the percent inhibition values of each titrated antagonist sample are used to calculate antagonist activity in units/ml.

Data showing the concentration needed to neutralize 40 pg/ml of IL-15 in a CTLL inhibition assay is provided in Table I below. Table II below shows the activity of IL-15 (agonist activity) and IL-15 antagonists in CTLL and CTLL inhibition assays.

TABLE I
Specific Activity of IL-15 Antagonists
The concentration of antagonist required to
neutralize 40 pg/ml IL-15 in CTLL inhibition assay:

| | | | method of |
|---|---|---|---|
| Antagonist | concentration | | protein determination |
| hull-15 minutes | 848-2560 | pg/ml | ELISA/estimated from AAA |
| M110, M111 | 5 | ng/ml | OD |
| PGEGhuIL-15 D56C | 7.7 | ng/ml | Estimated from AAA |
| M112 | 40 | ng/ml | OD |
| PEGf-s-IL15 | 140-196 | ng/ml | AAA |

OD = optical density absorbence at 280 nm; extinction coefficient of 1.35
AAA = amino acid analysis
PEGf-s-IL15 + PEGylated flag simian IL-15

TABLE II
Activity of IL-15 and IL-15 Antagonists
In CTLL and CTLL Inhibition Assays

| | CTLL Assay | CTLL Inhibition Assay |
|---|---|---|
| | units/ml | units/ml |
| sample | (Agonist Activity) | (Antagonist Activity) |
| IL-15 | 7.09 × 10⁵ | 279 |
| IL-15-Q156C | — | 3 × 10⁶ |
| IL-15-Q156S | — | 1.5 × 10⁶ |
| IL-15-D56C | — | 2 × 10⁶ |
| IL-15-D56C-Q156C | — | 7 × 10⁶ |
| IL-15-D56C-Q156S | — | 7.2 × 10⁵ |
| IL-15-D56S | — | 2.2 × 10⁵ |
| IL-15-D56S-Q156S | — | 7.2 × 10⁵ |
| vector control | — | 1141 |
| IL-15 | 3.7 × 10⁸ | |
| PEG-IL-15 | — | 2.3 × 10⁶ |
| PEG-IL-15-D56C | — | 7.96 × 10⁶ |
| IL-15-D56C | — | 5 × 10⁶ |
| IL-15 | 5.6 × 10⁸ | NA |
| PEG-IL-15 | NA | 1.7 × 10⁵ |

Q156C = Gln¹⁵⁶ substituted with Cys
Q156S = Gln¹⁵⁶ substituted with Ser
D56C = Asp⁵⁶ substituted with Cys
D56S = Asp⁵⁶ substituted with Ser
NA: not assayed

The antagonists according to the invention find use, as described above and in more detail below, in treating B cell tumors of GC origin, and conditions in which inhibition of B cell proliferation in the germinal center is desired.

As described above, another embodiment of the invention utilizes the nucleic acids that encode the IL-15 muteins of the invention. Such nucleic acids comprise either RNA or the cDNA having the nucleotide sequence from 144 to 486 of SEQ D NO:1 and 144 to 486 of SEQ ID NO:2. Further within the scope of the invention are expression vectors that comprise a cDNA encoding an IL-15 mutein and host cells transformed or transfected with such expression vector. Transformed host cells are cells that have been transformed or transfected with a recombinant expression vector using standard procedures. Expressed mammalian IL-15 will be located within the host cell and/or secreted into culture supernatant, depending upon the nature of the host cell and the gene construct inserted into the host cell. Pharmaceutical compositions comprising any of the above-described IL-15 antagonists also are encompassed by this invention.

Administration of Antagonists of IL-15. The present invention provides methods of using pharmaceutical compositions comprising an effective amount of IL-15 antagonist in a suitable diluent or carrier. An IL-15 antagonist of the invention can be formulated according to known methods used to prepare pharmaceutically useful compositions. An IL-15 antagonist can be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. In addition, such compositions can contain an IL-15 antagonist complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of an IL-15 antagonist. An IL-15 antagonist can also be conjugated to antibodies against tissue-specific receptors, ligands or antigens, or coupled to ligands of tissue-specific receptors.

The IL-15 antagonist of the invention can be administered topically, parenterally, rectally or by inhalation. The term “parenteral” includes subcutaneous injections, intravenous, intramuscular, intracisternal injection, or infusion techniques. These compositions will typically contain an effective amount of an IL-15 antagonist, alone or in combination with an effective amount of any other active material. Such dosages and desired drug concentrations contained in the compositions may vary depending upon many factors, including the intended use, patient's body weight and age, and route of administration. Preliminary doses can be determined according to animal tests, and the scaling of dosages for human administration can be performed according to art-accepted practices.

Preferably, anti-IL-15 antibodies are administered at low protein doses, such as 20 to 100 milligrams protein per dose, given once, or repeatedly, parenterally. Alternatively, anti-IL-15 antibodies are administered in doses of 30 to 90 milligrams protein per dose, or 40 to 80 milligrams protein per dose, or 50 to 70 milligrams protein per dose.

The anti-IL-15 antibody components, immunoconjugates, and fusion proteins of the present invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Remington's Pharmaceutical Sciences, 19th Ed. (1995).

For purposes of therapy, antibody components (or immunoconjugates/fusion proteins) and a pharmaceutically acceptable carrier are administered to a patient in a therapeutically effective amount. A combination of an antibody component, optionally with an immunoconjugate/fusion protein, and a pharmaceutically acceptable carrier is said to be administered in a “therapeutically effective amount” if the amount administered is physiologically significant. An agent is physiologically significant if its presence results in a detectable change in the physiology of a recipient patient. In the present context, an agent is physiologically significant if its presence results in the inhibition of the growth of target tumor cells.

For lymphoma treatment, inhibition of IL-15 stimulation of GC B cells may be carried out in conjunction with currently used antilymphoma therapy, including radiation therapy, chemotherapy, and/or biologic therapy. Biologic therapy generally is comprised of interferon therapy and monoclonal antibodies. Interferon therapy was the first biologic treatment studied in NHL. It is widely used in Europe for the treatment of indolent lymphomas, but it is seldom used in the United States. Data for the use of interferon maintenance therapy suggest prolonged disease-free survival but no consistent overall survival benefit (Hagenbeek, et al., Blood 92 (Suppl. 1:315a, 1998). The role for interferon therapy in patients with indolent lymphomas, therefore, remains under clinical evaluation. Thus, the IL-15 therapy described herein may be used as an adjunct to interferon therapy. Monoclonal antibodies are also in use for treating B cell lymphoma. Some monoclonal antibodies currently in use or under investigation in treatment of B cell lymphoma include Rituximab (Rituxan); CAMPATH-1H (Humanized IgG1); Tositumomab (Bexxarr); Ibritumomab tiuxetan (Zevalin); Epratuzumab; Bevacizumab; and Lym-1 (Oncolym). These therapies mainly target CD20, CD22, CD52, and VEGF (vascular endothelial growth factor). None of them specifically target IL-15 or IL-15-stimulated B cell growth in GC's.

The present invention, thus generally described, will be understood more readily by reference to the following examples, which are provided by way of illustration and are not intended to be limiting of the present invention.

### Examples

Material and Methods for Examples

Antibodies

Anti-IL-15 mAb (M110 and M111: IgG₁; M112: IgG_{2b}) were generated as described generally in U.S. Pat. No. 5,795,966. Briefly, Balb/c mice were boosted twice with 10 μg of human (h) IL-15-flag in RIBI adjuvant (Ribi Corp, Hamilton, Mont.). Three month after the last boost, one animal was boosted intravenously with 3 μg of hIL-15 in PBS. Three days later, the spleen was removed and fused with Ag8.653 using 50% PEG (Sigma, St. Louis, Mo.). The fused cells were plated into 96 well plates in DMEM containing HAT supplement (Sigma). Hybridoma supernatants were screened by antibody capture assay. Briefly, 96 well plates were coated with 10 μg/ml of goat anti-mouse Ig, overnight. After blocking with 3% BSA, 50 μl of cell supernatant were added to each well. After one hour plates were washed with PBS with 0.05% Tween 20. Iodinated hIL-15 was added to plates for 1 hour. After washing, plates were exposed to phosphoimager plates for three hours. Positive cells were cloned out twice, using similar screen to detect positives.

A CTLL-2 cell proliferation assay was also performed to determine IL-15 blocking activity. Specificity of these mAbs has been tested and used previously (U.S. Pat. No. 5,795,966; Tinhofer, I., et al. 2000. Blood 95:610. Musso, T., et al. 1999. Blood 93:3531). Mouse IgG₁ (MOPC 21) and IgG_{2b} (MOPC 141) for isotype control were obtained from Sigma. Anti-IL-15 mAb (MAB247, mouse IgG₁), goat polyclonal anti-IL-15, and goat normal control Ig were obtained from R&D systems (Minneapolis, Minn.). PE-conjugated anti-CD20 mAb and FITC-conjugated goat anti-mouse Ab were obtained from BD Pharmingen (San Diego, Calif.). DRC1 mAb (mouse IgG₁) were obtained from DAKO (Carpinteria, Calif.). Alexa 594-conjugated goat anti-mouse Ab was obtained from Molecular Probes (Eugene, Oreg.). FITC-conjugated donkey anti-goat Ab was obtained from Jackson ImmunoResearch Laboratories, Inc. (West Grove, Pa.).

Cytokines and Reagents

The culture medium used was IMDM (Irvine Scientific, Santa Ana, Calif.) and RPMI 1640 (Sigma) supplemented with 10% FCS (Life Technologies, Inc., Grand Island, N.Y.), 2 mM glutamine, 100 U/ml penicillin G, and 100 μg/ml streptomycin (Irvine Scientific). Cytokines used were IL-2 (Hoffman-La Roche, Inc., Nutley, N.J.), and IL-4 (Schering-Plough Schering Corporation, Union, N.J.). Recombinant trimeric human CD40 ligand (L) and IL-15 were prepared as described previously (Grabstein, K. H., et al. 1994. Science 264:965. Armitage, R. J., et al. 1995. J Immunology 154:483. Morris, A. E., et al. 1999. J Biol Chem 274:418.). Percoll and Ficoll were obtained from Pharmacia LKB Biotechnology (Uppsala, Sweden) and BSA from Sigma.

Immunofluorescence Staining of FDC Clusters

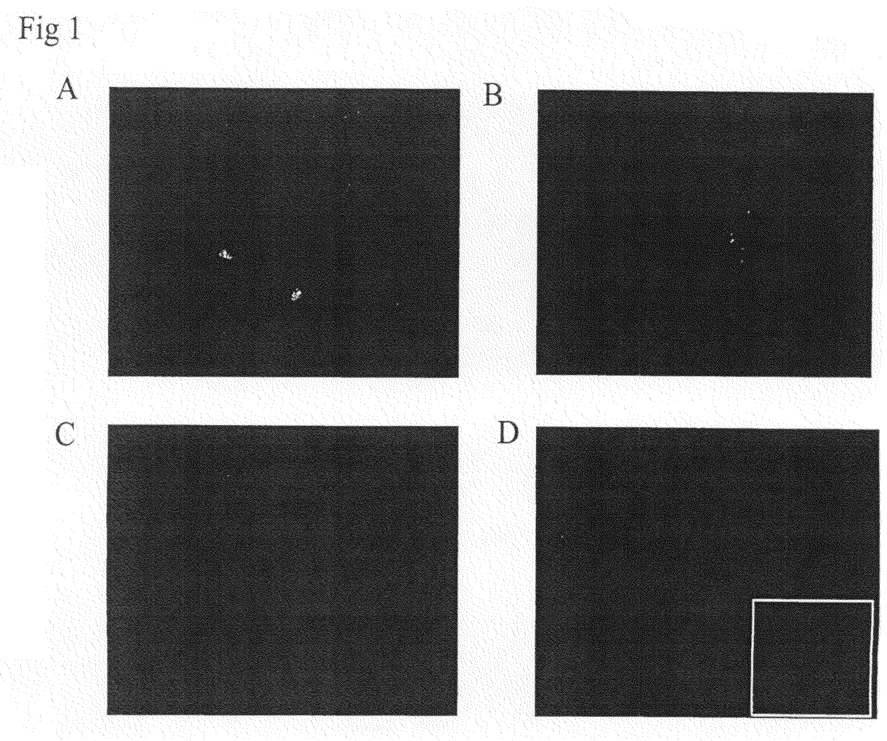

Human tonsillar FDCs were isolated as described previously (Kim, H.-S., et al. 1994. J. Immunology 153:2951.). Isolated cells were cytospun on glass slides at 700 rpm for 5 min (Cytosine 2®, Shandon, Pittsburgh, Pa.). The cytospin slides were fixed in cold acetone (−20° C.) for 5 min and stored at −70° C. until required. Slides were hydrated with PBS for 10 min at room temperature then incubated with blocking solution (DAKO) for 1 hour at 25° C. in a humidified chamber. Slides were stained with optimal amount of goat anti-IL-15 Ab or control goat Ig overnight at 4° C. The slides were then washed three times and incubated with FITC-conjugated anti-goat Ig for 1 h at room temperature. For costaining, DRC-1 mAb (FIGS. 1A and C) or PE-conjugated anti-CD20 mAb (for FIG. 1B) were added together with primary Abs. DRC-1 staining was visualized by secondary Alexa-594-conjugated anti-mouse Ab staining. For single FDC staining (FIG. 1D), slides were incubated in DAPI solution (Molecular Probes) for nuclear counter staining, then stained with mouse anti-IL-15 or control mAb followed by FITC-conjugated goat anti-mouse Ab. Slides were washed and mounted with anti-fade fluorescent mounting medium (Molecular Probes). Images were collected on a deconvolution microscope (Axiovert 200M; Carl Zeiss Microimaging, Inc., Thomwood, N.Y.). Images were processed using the slidebook software (version 1.6.587; Intelligent Imaging Innovations, Denver, Colo.) and Adobe Photoshop 7.0 (Adobe systems, Inc., San Jose, Calif.).

Flow Cytometric Analysis

FDC/HK cells were cultured in 10% FCS RPMI media as described previously (Kim, H.-S., et al. 1994. J. Immunology 153:2951). FDC/HK cells of passage 4-9 were used for the experiments. For FACS analysis, FDC/HK cells were collected with enzyme free cell dissociation solution (Specialty Media, Philipsburg, N.J.). All FACS staining for surface IL-15 detection was performed with modification to previously described procedures for amplification (Jung, J., et al. 2000. Eur. J. Immunology 30:2437). Briefly, cells were washed in cold FACS buffer (0.05% FCS, 0.01% NaN₃ in PBS) and subsequently incubated with the appropriate concentration of anti-IL-15 mAb (B247) for 15 min at 4° C. After washing with cold FACS buffer, the amplification procedures using Flow-Amp® kit (Flow-Amp systems, Cleveland, Ohio) were followed according to the manufacturer's instruction. For competition study, anti-IL-15 antibody was incubated with 300 ng/ml of recombinant IL-15 for 30 minutes at 4° C. prior to FACS staining. Samples were analyzed with FACSCalibur® (Becton Dickinson, San Jose, Calif.) and CellQuest-Pro® programs. Specific mean fluorescence intensity (MFI) was obtained by subtraction of fluorescence value from that of corresponding control.

Acid Stripping and Binding of IL-15

Acid stripping of previously bound IL-15 was performed as described (Dubois, S., et al. 2002. Immunity 17:537. Kumaki, S., et al. 1996. Eur J Immunology 26:1235.). Briefly, FDC/HK cells were washed twice with cold PBS, then incubated with glycine buffer (25 mM glycine, 150 mM NaCl, pH 3) for 10 min at 4° C. Cells were then collected and washed twice with cold PBS and subjected to FACS staining. For binding experiments, FDC/HK cells or GC-B cells were collected and washed with cold PBS twice, and then incubated with a saturating dose of IL-15 (100 ng/ml) for 30 min at 4° C., washed with cold PBS, and then stained for FACS analysis.

CTLL-2 Cell Assay

CTLL-2 cells (ATCC, Manasas, Va.) were maintained in RPMI 1640 media containing 10% FCS, IL-2 (30 U/ml) and 2-ME (5×10⁻⁵M, Sigma). Serially diluted numbers of FDC/HK cells (from 2×10⁴ cell/well to none/well) were cultured in 96 well plates for 1 day in a 5% CO₂ incubator. The plates were then washed and fixed in 1% paraformaldehyde in PBS for 1 hour at 4° C. followed by extensive washing in cold PBS. CTLL-2 cells (5×10³ cell/well) in maintaining media were added in triplicate to the 96 well plates coated with fixed FDC/HK cells and cultured with anti-IL-15 mAb or isotype control mAb. After 20 h of culture, cells were pulsed with 0.5 μCi of [³H] TdR (20 Ci/mM; PerkinElmer Life Sciences, Boston, Mass.) for additional 4 h. The cultures were harvested onto glass fiber filter and [³H] TdR incorporation was measured by a liquid scintillation counter (Rackbeta; LKB instrument, Houston, Tex.). Results are expressed as the mean cpm±SEM of triplicate cultures.

RT-PCR

To examine the expression of mRNA for IL-15Rα, IL-2Rα, IL-2Rβ, and IL-2Rγ, total RNA was extracted from cells using the RNeasy kit (Qiagen, Valencia, Calif.). One μg aliquot of RNA was transcribed using random oligo-dT and M-MLV RT (Invitrogen-Gibco, Carlsbad, Calif.). Complementary DNA was amplified in a 25 μl reaction mixture containing 200 μM of each dNTP, 500 nM of primers, and 2.5 U Taq polymerase. Amplification of each cDNA sample was carried out under condition as follows: denaturation at 94° C. for 50 sec, annealing at 57° C. for 50 sec, and extension at 72° C. for 50 sec. Human GAPDH was used to ensure equal sample loading. A mock PCR was performed to serve as a negative control. Amplified PCR products were separated on 1.5% agarose gel and visualized by ethidium bromide staining. Primers used are as follows: For IL-15Rα, 5′-GTCAAGAGCTACAGCTTGTAC-3′ (SEQ ID NO:3) and 5′CATAGGTGGTGAGAGCAGTTTTTC-3′ (SEQ ID NO:4); for IL-2Rα, 5′-AAGCTCTGCCACTCGGAACACAAC-3′ (SEQ ID NO:5) and 5′-TGATCAGCAGGAAAACACAGC-3′ (SEQ ID NO:6); for IL-2Rβ, 5′-ACCTCTTGGGCATCTGCAGC-3′ (SEQ ID NO:7) and 5′-CTCTCCAGCACTTCTAGTGG-3′ (SEQ ID NO:8); for IL-2Rγ, 5′-CCAGAAGTGCAGCCACTATC-3′ (SEQ ID NO:9) and 5′-GTGGATTGGGTGGCTCCAT-3′ (SEQ ID NO:10); and for GAPDH, 5′-CCCTCCAAAATCAAGTGGGG-3′ (SEQ ID NO:11) and 5′-CGCCACAGTTTCCCGGAGGG-3′ (SEQ ID NO:12).

Preparation and Culture of Human Tonsillar GC-B Cells

GC-B cells were purified from tonsillar B cells by MACS (Miltenyi Biotec Inc., Auburn, Calif.) as described (Choe, J., et al. 1996. J. Immunology 157:1006). The purity was greater than 95%, as assessed by the expression of CD20 and CD38. GC-B cells (2×10⁵ cell/well) were cultured in 24 well plates in the presence of irradiated FDC/HK cells (2×10⁴ cell/well, 5,000 Rad), CD40L (100 ng/ml), IL-2 (30 U/ml), and IL-4 (50 U/ml). IL-2 was included to increase sensitivity except for the experiment for FIG. 5B, since the overall recoveries of cultures were very low without IL-2 (Choe, J., et al. 1996. J. Immunology 157:1006). For blocking experiments, anti-IL-15 or isotype control mAb (10 μg/ml, unless indicated otherwise) was incubated for 30 min before adding GC-B cells. Some of blocking and corresponding control mAbs contained less than 0.00002% of sodium azide at working concentration, which is 100 fold lower than the concentration of sodium azide which started to show toxicity in the in vitro culture system. For addition experiments (FIG. 5B), IL-15 (1-100 ng/ml) was added 30 min before adding GC-B cells. For cell division experiments, GC-B cells were labeled with CFSE (Sigma, 5 μM/ml in PBS) at 37° C. for 10 min. FCS was added to stop staining, and then labeled cells were washed with culture media. After culture, the CFSE intensity was measured by FACSCalibur® and analyzed by ModFit LT®software 3.0 (Verity Software House, Inc. Topsham, Me.). Recovered viable cells were counted by trypan blue exclusion.

### Example 1

### IL-15 was Produced by FDC but not by B Cells

To identify the cellular source of IL-15 in the germinal centers, the in vivo expression of IL-15 was examined by staining freshly isolated FDC-B cell clusters with specific Abs to IL-15 (FIG. 1). FDC clusters were cellular aggregates consisting of a typical FDC with large cytoplasm and more than 10 B cells (Li, L., et al. 2000. Journal of Experimental Medicine 191:1077) (FIG. 1A-C). IL-15 was expressed in the FDC clusters, suggesting the presence of IL-15 in vivo (FIGS. 1A and B). To determine the cellular source of IL-15 in FDC clusters, FDC-specific marker DRC-1 mAb or B cell-specific marker anti-CD20 mAb was costained with goat anti-IL-15 Ab respectively (Li, L., et al. 2000. Journal of Experimental Medicine 191:1077. Naiem, M., et al. 1983. J. Clin. Pathol. 36:167.). Anti-IL-15 Ab (green) costained with DRC-1 mAb (red; costaining: yellow, FIG. 1A) but not with anti-CD20 mAb (red, FIG. 1B), suggesting that DRC-1 positive FDCs, not B cells, produce IL-15. The staining was specific for IL-15 since there was no costaining in samples costained with the goat control and DRC-1 Abs (FIG. 1C). Some FDCs (10-20%) were not clustered with B cells, but can be identified by their abundant cytoplasm and frequent double nuclei (van Nierop, K., et al. 2002. Semin Immunology 14:251) (FIG. 1D). These single FDCs also expressed IL-15 as stained by a murine anti-IL-15 mAb (MAB247), confirming the above result. Similarly, there was no green staining but only blue nuclear staining in samples stained with mouse control mAb and DAPI (1D-inset).

### Example 2

### IL-15 was Present on the Surface of FDC/HK Cells Bound to IL-15Rα

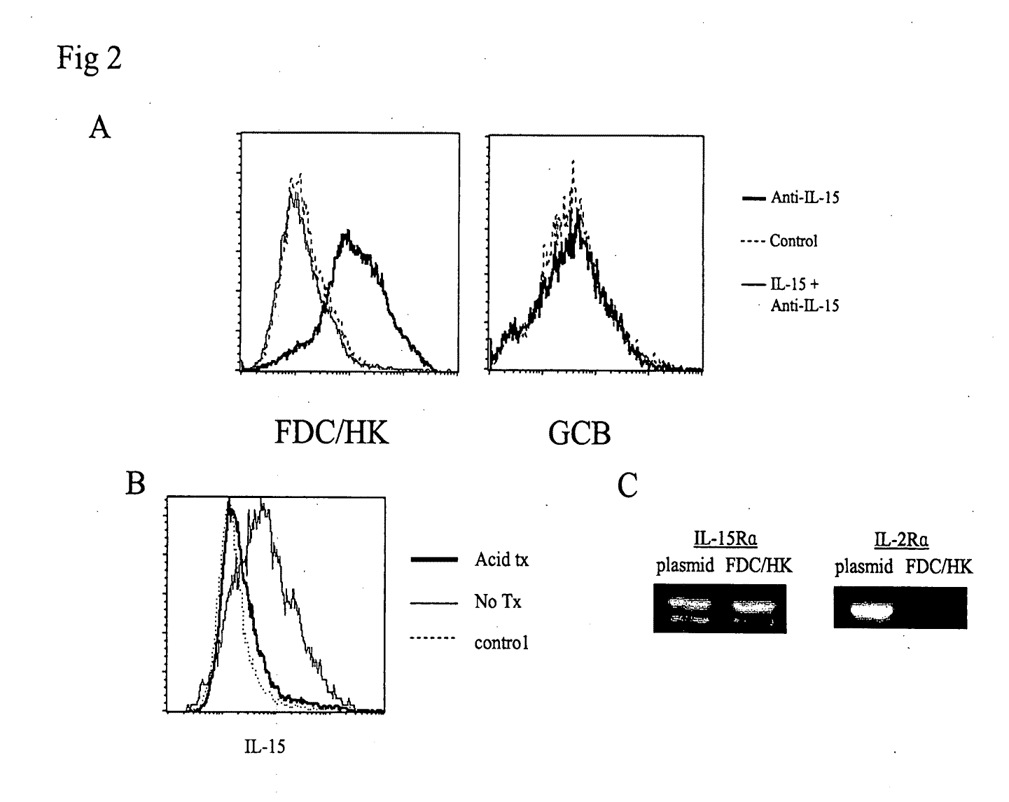

The production of IL-15 by a primary FDC cell line, FDC/HK, which was shown to share many of FDC characteristics including the capacity to support GC-B cell survival and proliferation (Li, L. et al., Semin. Immunol. 14:259, 2002; Kim, H.-S. et al., J. Immunol. 155:1101, 1995) was investigated. Because IL-15 was not detected in the culture supernatant of FDC/HK cells (2×10⁵ cells/ml) by ELISA (assay sensitivity≧19 pg/ml), surface expression of IL-15 was studied using methods as reported (Morris, A. E., et al. 1999. J Biol Chem 274:418; Kim, H.-S., et al. 1994. J. Immunology 153:2951; Naiem, M., et al. 1983. J. Clin. Pathol. 36:167; Bulfone-Paus, S., et al. 1997. Nat Med 3:1124). A highly sensitive surface FACS staining method using tyramine amplification method (Flow-Amp®) was used to detect IL-15. As shown in FIG. 2A, IL-15 was detected on FDC/HK cells whereas GC-B cells were negative (FIG. 2A). These results are consistent with the previous IF staining data on FDC-B cell clusters. The specific staining of IL-15 on FDC/HK was verified by competing with soluble IL-15. When anti-IL-15 mAb was preincubated with excess amount of IL-15, the staining of IL-15 on the surface of FDC/HK cells was completely reduced to that of isotype control. These results were reproduced in 3 separate experiments.

The surface IL-15 might have been due to the presence of an alternative membrane type IL-15 molecule (Musso, T., et al. 1999. Blood 93:3531), or through the rebinding of secreted IL-15 (Dubois, S., et al. 2002. Immunity 17:537. Schluns, K. S., et al. 2004. Blood 103:988.). Using acid treatment as described previously (Dubois, S., et al. 2002. Immunity 17:537), IL-15 was completely removed from the surface of FDC/HK cells after treatment with glycine buffer (pH 3.0) to the staining level with the control mAb (FIG. 2C). This result indicates rebinding of secreted IL-15 rather than an alternative membrane-type protein.

Because IL-15 Rα binds to IL-15 with high affinity (Giri, J. G., et al. 1995. Embo J 14:3654), the presence of IL-15 Rα in FDC/HK cells was examined. In RT-PCR experiments, the specific band for the IL-15 Rα was amplified from the cDNA of FDC/HK cells as well as positive control plasmid whereas that for the IL-2 Rα was not amplified, which was included to serve as an internal negative control (FIG. 2C). This result indicates that FDC/HK cells express mRNA for IL-15Rα.

### Example 3

### Membrane Bound IL-15 on the FDC/HK Surface is Biologically Active

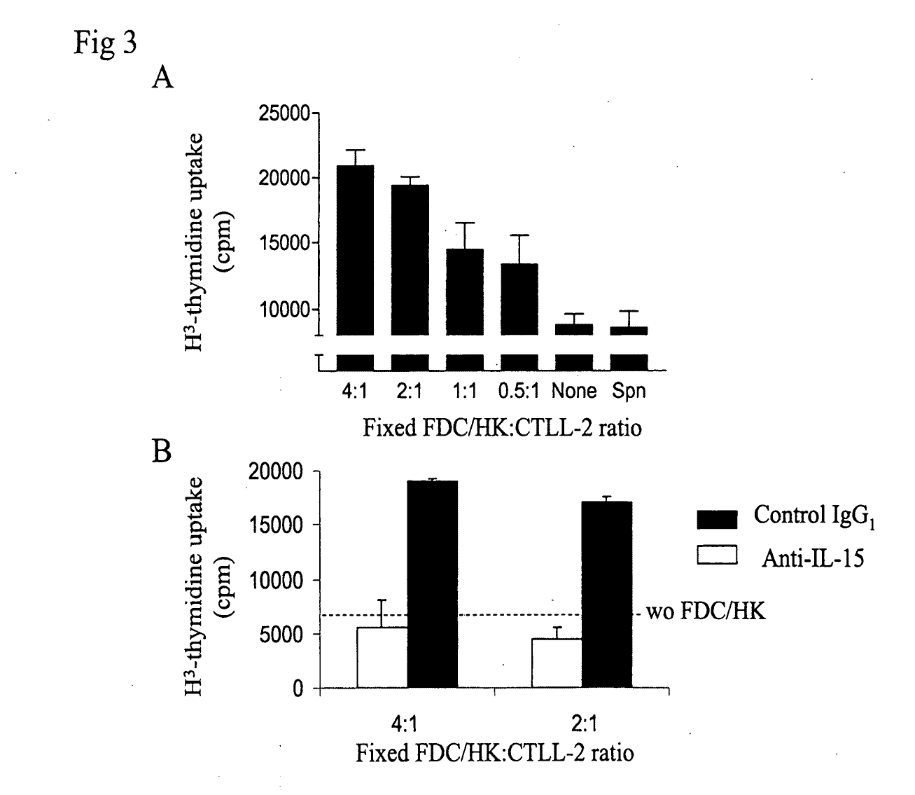

To examine the biological activity of surface bound IL-15 on FDC/HK cells, the IL-2 and IL-15 dependent CTLL-2 cell assay was employed. Although soluble IL-15 was not detectable by ELISA, FDC/HK cells were fixed with 1% paraformaldehyde to exclude the false positive results by soluble IL-15. Incorporation of tritiated thymidine by CTLL-2 cells increased in proportion to the number of fixed FDC/HK cells present in cultures (FIG. 3A). At the ratio of 4:1 of FDC/HK cells to responding CTLL-2 cells, the value of cpm was almost three times higher than negative controls (21,000 to 7,500). The relatively higher background proliferation of CTLL-2 cells (7,500 cpm) without fixed FDC/HK cell control wells can be attributed to suboptimal dose of IL-2 added to increase the sensitivity of the assay. The result is consistent with the previous report that the rebound IL-15 is functionally active on the cell surface (Morris, A. E., et al. 1999. J. Biol Chem 274:418. Kim, H.-S., et al. 1994. J. Immunology 153:2951. Naiem, M., et al. 1983. J. Clin. Pathol. 36:167. Bulfone-Paus, S., et al. 1997. Nat Med 3:1124.). To examine the possible effect of soluble IL-15 released from the FDC/HK cells, the culture supernatant from the highest FDC/HK cell concentration (2×10⁴/well) was added to the same culture. There was no significant difference in cpm values between cultures with control media and with FDC/HK cell-culture supernatant, indicating the absence of IL-15 in the culture supernatant, which is consistent with the ELISA results.

To confirm that the stimulatory effect on CTLL-2 cells was mediated by IL-15, specific blocking mAb to IL-15 and isotype control mAb were added to the culture. As shown in FIG. 3B, the addition of anti-IL-15 mAb blocked completely the proliferation of CTLL-2 cells enhanced by fixed FDC/HK cells whereas the control mAb had no effect.

### Example 4

### GC-B Cells Express Receptor Components for IL-15 Signal Transduction but not for High Affinity Binding

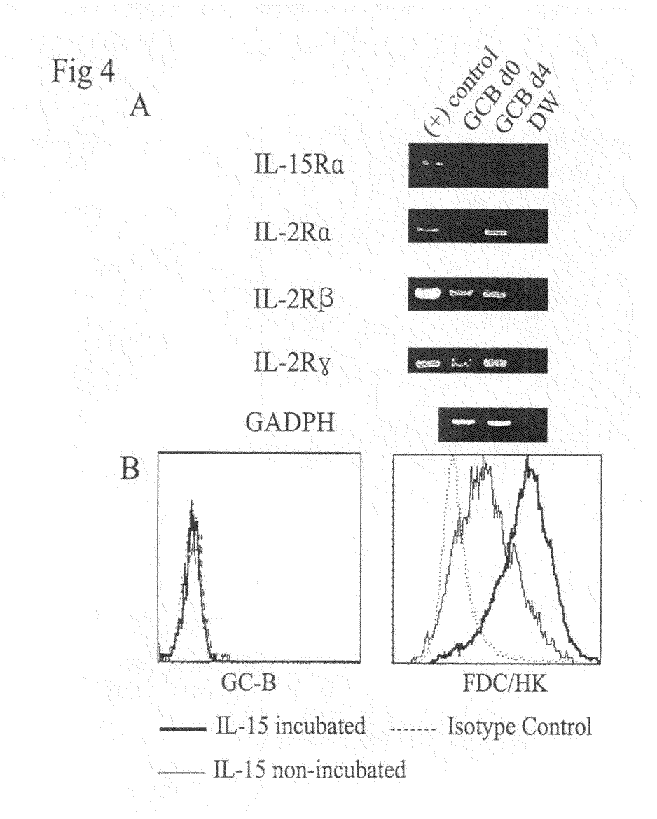

Production of IL-15 by FDC implied that IL-15 possibly had a biologic function in the GC reaction, most likely on GC-B cells. We thus examined the expression profile of specific receptors required for IL-15 signaling in GC-B cells (FIG. 4A). The expression of IL-15 Rα mRNA, a receptor component for high affinity binding, was virtually negligible in RT-PCR, showing a similar faint band to that of IL-2 Rα in freshly isolated GC-B cells (a negative control). In contrast, expressions of IL-2Rβ and IL-2Rγ mRNAs, the major components of signal transduction, were evident in GC-B cells whether freshly isolated or cultured, suggesting the presence of signaling receptor components for IL-15 or IL-2 in GC-B cells both in vivo and in vitro.

The absence of IL-15Rα mRNA was also confirmed by the failure to detect IL-15Rα protein in FACS staining of GC-B cells and the lack of IL-15 binding (FIG. 4B). In contrast to FDC/HK cells that exhibited intense binding of IL-15, no significant binding of IL-15 was detected on the surface of GC-B cells after incubation with excess IL-15, demonstrating the absence of IL-15Rα on the surface. Since soluble IL-15 needs IL-15α to transducer its mitogenic signal (Lu, J. et al., Clin. Cancer Res. 8:3877, 2002), the results suggest that GC-B cells cannot respond to soluble IL-15. This conclusion is consistent with the observation that soluble IL-15 in the absence of FDC-HK cells showed no noticeable difference in GC-B cell recovery.

### Example 5

### IL-15 Increases GC-B Cell Proliferation

GC-B cells were cultured with FDC/HK cells and cytokines as described above. When different amounts of anti-IL-15 mAb were added, GC-B cell proliferation was remarkably inhibited in a dose dependent manner (FIG. 4A), suggesting that IL-15 enhanced GC-B cell proliferation. At day 10, the number of viable GC-B cells in the culture containing anti-IL-15 mAb (10 μg/ml) was 17% of that of cultures containing isotype control mAb. However, blocking of IL-15 did not affect differentiation of cultures cells measured by surface marker and Ig secretion. This result was reproduced in four separate experiments. Similar inhibition was also observed in the experiments using other mAbs to IL-15 (Clone M111, M112 and MAB247).

In other experiments, IL-2 was omitted to exclude possible indirect effect by IL-2, and to verify the effect of IL-15 in the depletion experiment. As shown in FIG. 4B, the amount of surface IL-15 on FDC/HK cells was increased further by the incubation with exogenous IL-15. Hence, coated FDC/HK cells were incubated with different amount of IL-15 (1-100 ng) prior to GC-B cell cultures to augment IL-15 effect. The MFI of surface IL-15 by FACS were increased in proportion to the IL-15 added (for 100 ng: FIG. 4B right panel). The cell number recovered at culture day 10 was increased in a dose-dependent manner (FIG. 5B). In the presence of 100 ng/ml of IL-15, the number of viable GC-B cells increased two and half times more than the control culture. Given that GC-B cells do not express IL-15Rα, these results strongly suggested that surface IL-15 on FDC/HK enhanced GC-B cell proliferation. This result was reproduced in four separate experiments.

Throughout this application, various publications are referenced. The disclosures of these publications are hereby incorporated by reference herein in their entireties. The foregoing written description is considered to be sufficient to enable one skilled in the art to practice the invention. The present invention is not to be limited in scope by the examples presented herein. Indeed, various modifications of the invention in addition to those shown and described here will become apparent to those skilled in the art from the foregoing description and fall within the scope of the appended claims.

1. A method for treating a B-cell tumor of germinal center origin, comprising administering to a human subject having said B-cell tumor a therapeutic composition comprising a pharmaceutically acceptable carrier and at least one antagonist of IL-15.

2. The method of claim 1, wherein said antagonist is an anti-IL-15 antibody.

3. The method of claim 2, wherein said anti-IL-15 antibody is selected from the group consisting of non-human primate antibody, murine monoclonal antibody, chimeric antibody, human antibody, and humanized antibody.

4. The method of claim 2, wherein said anti-IL-15 antibody is parenterally administered in a dosage of 30-90 milligrams protein per dose.

5. The method of claim 2, wherein said subject receives anti-IL-15 antibody as repeated parenteral dosages of 50-90 milligrams protein per dose.

6. The method of claim 2, wherein said anti-IL-15 antibody is selected from the group consisting of antibodies M110, M111 and M112.

7. The method of claim 1, where said antagonist is a mutein of IL-15.

8. The method of claim 7, wherein said IL-15 mutein is capable of binding to the IL-15Rα. subunit, and is incapable of transducing a signal through the β- or γ-subunits of the IL-15 receptor complex.

9. The method of claim 7, wherein in said mutein, at least one of the amino acid residues Asp⁵⁶ or Gln¹⁵⁶ of IL-15 of SEQ ID NO:2 is deleted or substituted with a different naturally-occurring amino acid residue.

10. The method of claim 7, where said mutein is conjugated to a chemical moiety.

11. The method of claim 10, wherein said mutein is conjugated to polyethylene glycol.

12. The method of claim 1, wherein said antagonist is soluble IL-15.

13. The method of claim 12, wherein said soluble IL-15 is conjugated to a chemical moiety.

14. The method of claim 13, wherein said soluble IL-15 is conjugated to polyethylene glycol.

15. The method of claim 1, wherein said B-cell tumor is selected from the group consisting of Hodgkin's lymphoma; non-Hodgkin's lymphoma; B-cell lymphomas; small lymphocytic lymphoma; mantle cell lymphoma; follicular lymphoma; marginal cell lymphoma; monocytoid B-cell, lymphoma; splenic lymphoma; diffuse large cell lymphoma; Burkitt's lymphoma; high grade Burkitt-like lymphoma; lymphoblastic lymphoma; and diffuse large cell lymphoma

16. The method of claim 15, wherein said B-cell tumor is a non-Hodgkin's lymphoma.

17. The method of claim 1, further comprising administering a therapeutic protein or chemotherapeutic treatment, wherein said therapeutic protein is selected from the group consisting of antibody, immunoconjugate, antibody-immunomodulator fusion protein and antibody-toxin fusion protein.

18.-20. (canceled)

21. The method of claim 17, wherein said chemotherapeutic treatment consists of the administration of at least one drug selected from the group consisting of cyclophosphamide, etoposide, vincristine, procarbazine, prednisone, carmustine, doxorubicin, methotrexate, bleomycin, dexamethasone, phenyl butyrate, brostatin-1 and leucovorin.

22. The method of claim 1, wherein said therapeutic composition further comprises a cytokine moiety, wherein said cytokine moiety is selected from the group consisting of interleukin-1 (IL-1), IL-2, IL-3, IL-6, IL-10, IL-12, interferon-γ, interferon-β, and interferon-γ.

23. The method of claim 22, wherein said therapeutic protein is a immunoconjugate or antibody-toxin fusion protein that comprises a toxin selected from the group consisting of ricin, abrin, ribonuclease, DNase I, Staphylococcal enterotoxin-A, pokeweed antibiral protein, gelonin, diphtherin toxin, Pseudomonas exotoxin, and Pseudomonas endotoxin.

24.-25. (canceled)
